(19) United States
(12) Patent Application Publication
(10) Pub. No.: US 2020/0126391 A1
(43) Pub. Date: Apr. 23, 2020

(54) Drowning Prevention System

(71) Applicant: Nicole Lovett, Pompano Beach, FL (US)

(72) Inventor: Nicole Lovett, Pompano Beach, FL (US)

(21) Appl. No.: 16/658,213

(22) Filed: Oct. 21, 2019

Related U.S. Application Data
(60) Provisional application No. 62/748,713, filed on Oct. 22, 2018.

(51) Int. Cl. G08B 21/08; A61B 5/024; A61B 5/00; G08B 25/10; H04W 4/029

(52) U.S. Cl. CPC G08B 21/088; A61B 5/024; H04W 4/029; G08B 25/10; A61B 5/6824

(57) ABSTRACT

A drowning prevention system. The drowning prevention system includes a housing having a strap affixed to opposing ends thereof. The strap removably secures the housing to a user, such that a heartbeat sensor connected to a pair of contacts on the housing is in contact with the user. A moisture sensor is also disposed within the housing. An alert signal is sent to a mobile device via a wireless transceiver disposed within the housing when either the moisture sensor or the heartbeat sensor detects a predefined abnormal situation, such as heartrates above a threshold heartrate for an extended period or upon detection of the housing being submerged in a volume of liquid.

### CROSS REFERENCE TO RELATED APPLICATIONS

This application claims the benefit of U.S. Provisional Application No. 62/748,713 filed on Oct. 22, 2018. The above identified patent application is herein incorporated by reference in its entirety to provide continuity of disclosure.

### BACKGROUND OF THE INVENTION

The present invention relates to drowning prevention systems. More particularly, the present invention pertains to a drowning prevention system that detects whether a user's heartbeat is outside a range of acceptable heartrates and whether the drowning prevention system is submerged in a body of liquid and sends an alert signal to a mobile device in these situations.

Many people enjoy swimming, however swimming alone can involve additional risk of drowning, as bystanders are not readily available to contact emergency services if a person is drowning. Additionally, children or distracted individuals may wander into a pool or other body of water without a guardian or the like noticing, which could further lead to severe risk of drowning. During drowning events, the victims typically suffer from elevated or otherwise irregular heartrates, which could lead to further complications without immediate medical assistance. Therefore, a device that can immediately contact emergency services upon detection of a user's irregular or elevated heartrate, or upon the detection of a user being completely submerged in a body of water, is desired.

In light of the devices disclosed in the known art, it is submitted that the present invention substantially diverges in design elements from the known art and consequently it is clear that there is a need in the art for an improvement to existing drowning prevention systems. In this regard, the instant invention substantially fulfills these needs.

### SUMMARY OF THE INVENTION

In view of the foregoing disadvantages inherent in the known types of drowning prevention systems now present in the known art, the present invention provides a drowning prevention system wherein the same can be utilized for providing convenience for the user when alerting a user and third parties, including emergency services, of a potential drowning incident.

The present system comprises a housing having a strap affixed to opposing ends thereof, wherein the strap removably secures the housing to a user. A heartbeat sensor is disposed within the housing, wherein the heartbeat sensor is operably connected to a pair of contacts disposed on a rear side of the housing. The heartbeat sensor is configured to monitor a heartrate of a user in contact with the pair of contacts. A moisture sensor is disposed within the housing, wherein the moisture sensor is configured to determine when the housing is submerged in a liquid. A wireless transceiver is disposed within the housing, wherein the wireless transceiver is operably connected to the heartbeat sensor and the moisture sensor. The wireless transceiver is configured to send an alert signal to a mobile device when the heartbeat sensor detects a heartrate above a threshold heartrate for a set amount of time. The wireless transceiver is also configured to send an alert signal to a mobile device when the moisture sensor determines the housing is submerged in a liquid for a set amount of time. In some embodiments, the strap is adjustable in length. In another embodiment, a width of the strap tapers from a proximal end thereof towards a distal end thereof. In other embodiments, the width of the proximal end is equal to a width of the housing, such that an upper surface of the strap and a lower surface of the strap are flush with a front side of the housing and the rear side. In yet another embodiment, a display is disposed on a front side of the housing, wherein the display is configured to display the heartrate detected by the heartbeat sensor. In some embodiments, a GPS unit is disposed within the housing, wherein the GPS unit is configured to determine the location of the housing. In another embodiment, the wireless transceiver is configured to send the location determined by the GPS unit to the mobile device when the alert signal is sent. In other embodiments, an indicator light is disposed on the front side of the housing, wherein the indicator light is configured to illuminate when the heartbeat sensor detects a heartrate above the threshold heartrate. In yet another embodiment, the indicator light is further configured to illuminate when the moisture sensor determines that the housing is submerged within a liquid. In some embodiments, the indicator light is disposed about a periphery of the front side. In some embodiments, a vibration motor is disposed within the housing, wherein the vibration motor is configured to activate when the heartbeat sensor detects a heartrate above the threshold heartrate. In another embodiment, the vibration motor is further configured to activate when the moisture sensor determines that the housing is submerged in a liquid.

A method of using a drowning prevention system comprises providing a housing having a heartbeat sensor and a moisture sensor therein, detecting a heartrate of a user in contact with a pair of contacts disposed on a rear side of the housing, comparing the heartrate with a predetermined threshold heartrate stored on a memory within the housing, sending an alert signal to a mobile device via a wireless transceiver disposed within the housing when the heartrate exceeds the threshold heartrate for a set length of time, determining whether the housing is submerged within a liquid via the moisture sensor, and sending an alert signal to a mobile device via the wireless transceiver when the moisture sensor determines that the housing is submerged in a liquid for a set length of time. In some embodiments, the method further comprises activating an indicator light disposed on a front side of the housing when the heartbeat sensor detects a heartrate exceeding the threshold heartrate. In another embodiment, the method further comprises activating the indicator light when the moisture sensor determines that the housing is submerged in liquid. In other embodiments, the method further comprises activating a vibration motor disposed within the housing when the heartbeat sensor detects a heartrate exceeding the threshold heartrate. In yet another embodiment, the method includes activating the vibration motor when the moisture sensor determines that the housing is submerged in liquid. In some embodiments, the method includes sending a location determined by a GPS unit disposed within the housing when the alert signal is sent. In another embodiment, the method includes sending the alert signal to emergency services. In other embodiments, the method further comprises displaying a current heartrate detected by the heartbeat sensor on a display disposed on the front side of the housing.

### BRIEF DESCRIPTION OF THE DRAWINGS

Although the characteristic features of this invention will be particularly pointed out in the claims, the invention itself and manner in which it may be made and used may be better understood after a review of the following description, taken in connection with the accompanying drawings wherein like numeral annotations are provided throughout.

FIG. 1 shows a perspective view of an embodiment of the drowning prevention system.

FIG. 2 shows a perspective view of an embodiment of the drowning prevention system.

FIG. 3 shows a schematic view of an embodiment of the drowning prevention system.

FIG. 4 shows a flow diagram of an embodiment of the drowning prevention system.

### DETAILED DESCRIPTION OF THE INVENTION

Reference is made herein to the attached drawings. Like reference numerals are used throughout the drawings to depict like or similar elements of the drowning prevention system. The figures are intended for representative purposes only and should not be considered to be limiting in any respect.

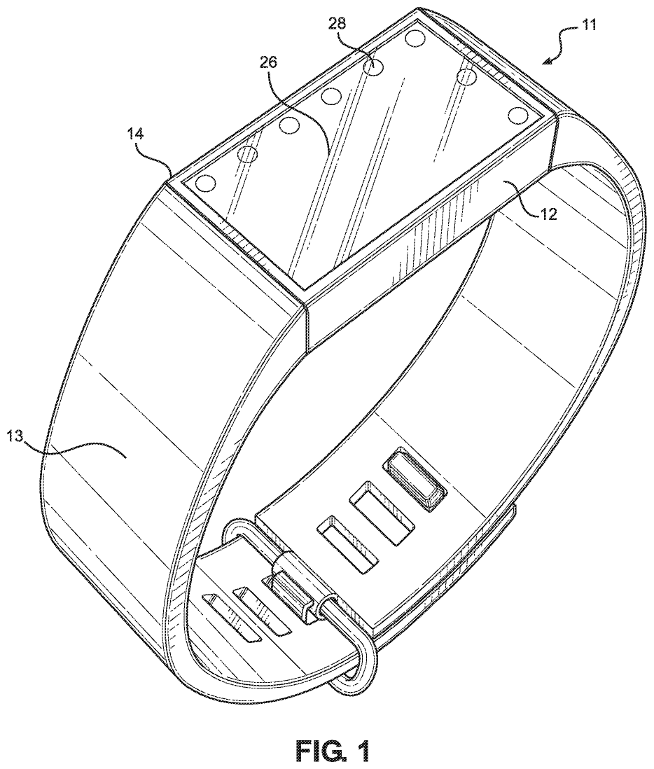

Referring now to FIG. 1, there is shown a perspective view of an embodiment of the drowning prevention system. The drowning prevention system 11 comprises a housing 12 having a strap 13 extending from opposing ends 14 of the housing 12. The housing 12 includes a variety of electronic components therein for the purposes of alerting a user and others to the wearer of the drowning prevention system 11 of a potential drowning event. These electronic components will be further described elsewhere in this disclosure. In the illustrated embodiment, the housing 12 is removably securable about a user's wrist via the strap 13, however, in alternate embodiments, the housing 12 is secured to a necklace strap, such that the housing 12 rests against a user's chest. In the illustrated embodiment, the strap 13 is adjustable via a buckle system, so as to allow securement to user's of various sizes, however, alternate adjustment systems are contemplated, including hook and loop fasteners along a length of the strap 13, elastically adjustable straps 13, and the like.

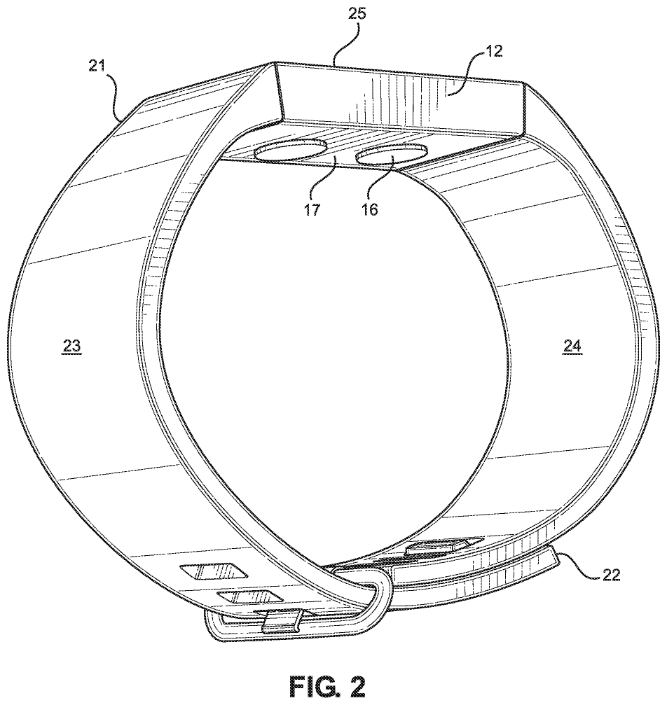

In the illustrated embodiment, the housing 12 further comprises a display 26 disposed on a front side (as shown in FIG. 2, 25) thereof. The display 26 can be configured to show a heartrate of the user thereon, in addition to showing additional information, such as the time, date, and the like thereon. In the shown embodiment, an indicator light 28 is disposed on the housing 12, wherein the indicator light 28 is configured to illuminate upon various conditions to be further defined elsewhere in this disclosure. In the illustrated embodiment, the indicator light 28 comprises a plurality of light sources disposed about a periphery of the housing 12, such that the indicator light 28 is readily visible from various viewing angles. In this way, the visibility of the indicator light 28 can be maximized.

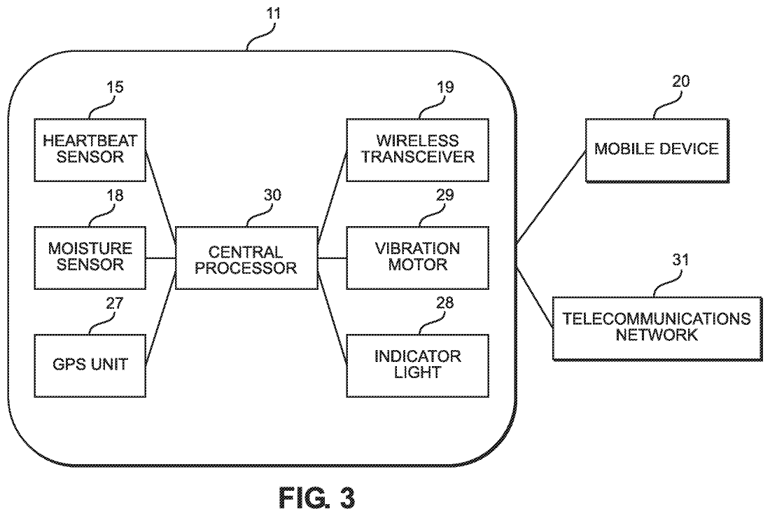

Referring now to FIG. 2, there is shown a perspective view of an embodiment of the drowning prevention system. A pair of contacts 16 are disposed on a rear side 17 of the housing 12, wherein the pair of contacts 16 are operably connected to a heartbeat sensor (as shown in FIG. 3, 15). Positioned along the rear side 17 of the housing 12, the pair of contacts 16 are configured to rest flush against a user, such that the pair of contacts 16 biometrically interact with a user, to detect a current heartrate of the user. In embodiments utilizing a necklace configuration, the pair of contacts 16 placement on the rear side 17 of the housing 12 allow the pair of contacts 16 to maintain direct contact with the user's chest. The current heartrate can then be displayed on the display as previously discussed.

In the illustrated embodiment, the straps 13 each taper inwardly from a proximal end 21 thereof towards a distal end 22 thereof. In this way, a width of the strap 13 decreases from the proximal end 21 towards the distal end 22. In the shown embodiment, an upper surface 23 of the strap 13 and a lower surface 24 of the strap 13 are flush with the front side 25 of the housing 12 and the rear side 17 of the housing 12, respectively. In this way, protrusions from the housing 12 are minimized, thereby reducing the likelihood of the housing 12 becoming caught or hung on an obstacle. In situations in which it is possible for a user to drown, such as swimming in a pool, such obstructions becoming caught on obstacles in the user's surroundings (i.e., an underwater obstacle) could themselves be the cause of drowning.

Referring now to FIG. 3, there is shown a schematic view of an embodiment of the drowning prevention system. The drowning prevention system 11 comprises a heartbeat sensor 15 and a moisture sensor 18. The heartbeat sensor 15 is operably connected to the pair of contacts and is configured to detect a heartrate of a user in contact with the pair of contacts. As a user's heartrate elevates in times of extreme stress, such as would accompany a drowning event, the detected heartrate can be used as one signal for the occurrence of a drowning event. Additionally, the moisture sensor 18 is disposed within the housing and is configured to detect when the housing is completely submerged in a liquid, such as water. In some embodiments, the moisture sensor 18 may additionally utilize a pressure sensor to determine depth of submersion of the housing. As the various other electronic components shown in the illustrated embodiment may have a negative relationship with water or other such liquids, it is advantageous to separate the moisture sensor 18 from the remaining electronic components within a waterproof sealed compartment. In this way, the exposure of the moisture sensor 18 to water does not impede the operation of the remaining electronic components. In the illustrated embodiment, the drowning prevention system 11 further comprises a GPS unit 27 configured to determine the present location of the housing, such that the location of the housing can be transmitted with an alert signal.

In the illustrated embodiment, a central processor 30 is operably connected to each of the electronic components, the central processor 30 configured to determine whether values detected by the various sensors exceed preset threshold values. The heartbeat sensor 15 is then operably connected to the central processor 30, wherein the central processor 30 is configured to compare the heartrate detected by the heartbeat sensor 15 to a predetermined threshold heartrate stored within a memory thereon. Furthermore, the central processor 30 is configured to detect whether the moisture sensor 18 continuously detects that the housing is submerged within a liquid for a preset length of time. Should the housing be continuously submerged for a prolonged period of time, the likelihood of a drowning event is high.

In some embodiments, each of the heartrate sensor 15 and the moisture sensor 18 must both detect readings beyond the threshold values for the central processor 30 to send an alert signal, however, in alternate embodiments, either sensor 15, 18 detecting such a reading can trigger the alert signal. Upon triggering the alert signal, an alert is sent to a mobile device 20 via a wireless transceiver 19 disposed within the housing of the drowning prevention system 11. In this way, a previously designated recipient, such as a friend, parent, guardian, or the like, can receive an alert, such as a text message, in the event the drowning prevention system 11 detects a potential drowning event. In some embodiments, the alert is also sent across a telecommunications network 31 to alert emergency services of a potential drowning event. In embodiments utilizing a GPS unit 27, the location of the housing can be sent to each recipient as well. In this way, an ambulance can be dispatched immediately to the location of the drowning event.

In some embodiments, the alert signal simultaneously activates the indicator light 28, such that a user or bystanders nearby are alerted to the potential drowning event. Additionally, in some embodiments, a vibration motor 29 is disposed within the housing, wherein the vibration motor 29 is configured to vibrate the housing when actuated. In embodiments having a vibration motor 29, the alert signal is configured to simultaneously actuate the vibration motor 29, alerting the wearer to the dangerous situation.

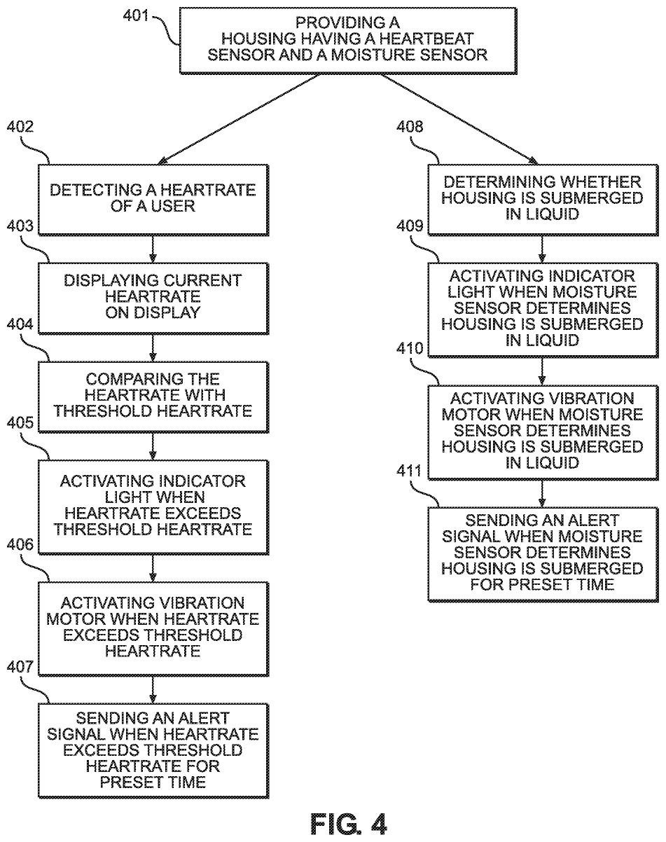

Referring now to FIG. 4, there is shown a flow diagram of an embodiment of the drowning prevention system. The method of using the drowning prevention device comprises providing 401 a housing having a heartbeat sensor and a moisture sensor. The heartbeat sensor detects 402 a heartrate of a user in contact with the housing. In some embodiments, the heartrate can be displayed 403 on a display disposed on the housing. The detected heartrate is then compared 404 with a threshold heartrate on the device. In some embodiments, should the detected heartrate exceed the threshold heartrate, an indicator light can be activated 405, alerting a user to the excessive heartrate. Additionally, in some embodiments, when the detected heartrate exceeds the threshold heartrate, a vibration motor within the housing is activated 406, further alerting the wearer of the elevated heartrate. Should the detected heartrate exceed the threshold heartrate for a preset length of time, an alert signal is sent 407 to a mobile device, emergency services, or both, thereby alerting others of the high likelihood of a drowning event. In the illustrated embodiment, the indicator light and the vibration motor are activated 405, 406 at any time the detected heartrate exceeds the threshold heartrate, however, the alert signal is only sent 407 when the heartrate exceeds the threshold heartrate for a particular length of time. In this way, momentary triggering of the heartrate sensor does not result in an alert signal, thereby reducing the number of false positives.

Similarly, the moisture sensor determines 408 whether the housing is submerged in a liquid, such as a body of water. In some embodiments, when the moisture sensor determines the submersion of the housing within a liquid, the indicator light is activated 409. In further embodiments, when the moisture sensor determines the submersion of the housing within a liquid, the vibration motor is activated 410. Upon determining the housing is submerged within a liquid for a preset length of time, an alert signal is sent 411 to a mobile device, emergency services, or both, thereby alerting others of the high likelihood of a drowning event. In the illustrated embodiment, the indicator light and the vibration motor are activated 409, 410 at any time the housing is determined to be submerged in a liquid, however, the alert signal is only sent 411 when the housing is determined to be submerged in a liquid for a particular length of time. In this way, momentary triggering of the moisture sensor does not result in an alert signal, thereby reducing the number of false positives. In some embodiments, both the moisture sensor and the heartbeat sensor must be triggered before the alert is sent.

It is therefore submitted that the instant invention has been shown and described in various embodiments. It is recognized, however, that departures may be made within the scope of the invention and that obvious modifications will occur to a person skilled in the art. With respect to the above description then, it is to be realized that the optimum dimensional relationships for the parts of the invention, to include variations in size, materials, shape, form, function and manner of operation, assembly and use, are deemed readily apparent and obvious to one skilled in the art, and all equivalent relationships to those illustrated in the drawings and described in the specification are intended to be encompassed by the present invention.

Therefore, the foregoing is considered as illustrative only of the principles of the invention. Further, since numerous modifications and changes will readily occur to those skilled in the art, it is not desired to limit the invention to the exact construction and operation shown and described, and accordingly, all suitable modifications and equivalents may be resorted to, falling within the scope of the invention.

I claim:

1. A drowning prevention system, comprising:
a housing having a strap affixed to opposing ends thereof;
wherein the strap removably secures the housing to a user;
a heartbeat sensor disposed within the housing, the heartbeat sensor operably connected to a pair of contacts disposed on a rear side of the housing;
wherein the heartbeat sensor is configured to monitor a heartrate of a user in contact with the pair of contacts;
a moisture sensor disposed within the housing, wherein the moisture sensor is configured to determine when the housing is submerged in a liquid;
a wireless transceiver disposed within the housing, the wireless transceiver operably connected to the heartbeat sensor and the moisture sensor;
wherein the wireless transceiver is configured to send an alert signal to a mobile device when the heartbeat sensor detects a heartrate above a threshold heartrate for a set amount of time;
wherein the wireless transceiver is configured to send an alert signal to a mobile device when the moisture sensor determines the housing is submerged in a liquid for a set amount of time.

2. The drowning prevention system of claim 1, wherein the strap is adjustable in length.

3. The drowning prevention system of claim 1, wherein a width of the strap tapers from a proximal end thereof towards a distal end thereof.

4. The drowning prevention system of claim 3, wherein the width of the proximal end is equal to a width of the housing, such that an upper surface of the strap and a lower surface of the strap are flush with a front side of the housing and the rear side.

5. The drowning prevention system of claim 1, further comprising a display disposed on a front side of the housing, wherein the display is configured to display the heartrate detected by the heartbeat sensor.

6. The drowning prevention system of claim 1, further comprising a GPS unit disposed within the housing, wherein the GPS unit is configured to determine the location of the housing.

7. The drowning prevention system of claim 6, wherein the wireless transceiver is configured to send the location determined by the GPS unit to the mobile device when the alert signal is sent.

8. The drowning prevention system of claim 1, further comprising an indicator light disposed on the front side of the housing, wherein the indicator light is configured to illuminate when the heartbeat sensor detects a heartrate above the threshold heartrate.

9. The drowning prevention system of claim 8, wherein the indicator light is further configured to illuminate when the moisture sensor determines that the housing is submerged in a liquid.

10. The drowning prevention system of claim 8, wherein the indicator light is disposed about a periphery of the front side.

11. The drowning prevention system of claim 1, further comprising a vibration motor disposed within the housing, wherein the vibration motor is configured to activate when the heartbeat sensor detects a heartrate above the threshold heartrate.

12. The drowning prevention system of claim 11, wherein the vibration motor is further configured to activate when the moisture sensor determines that the housing is submerged in a liquid.

13. A method of alerting a user to a drowning event, comprising:
providing a housing having a heartbeat sensor and a moisture sensor therein;
detecting a heartrate of a user in contact with a pair of contacts disposed on a rear side of the housing;
comparing the heartrate with a predetermined threshold heartrate stored on a memory within the housing;
sending an alert signal to a mobile device via a wireless transceiver disposed within the housing when the heartrate exceeds the threshold heartrate for a set length of time;
determining whether the housing is submerged within a liquid via the moisture sensor;
sending an alert signal to a mobile device via the wireless transceiver when the moisture sensor determines that the housing is submerged in a liquid for a set length of time.

14. The drowning prevention system of claim 13, further comprising activating an indicator light disposed on a front side of the housing when the heartbeat sensor detects a heartrate exceeding the threshold heartrate.

15. The drowning prevention system of claim 14, further comprising activating the indicator light when the moisture sensor determines that the housing is submerged in liquid.

16. The drowning prevention system of claim 13, further comprising activating a vibration motor disposed within the housing when the heartbeat sensor detects a heartrate exceeding the threshold heartrate.

17. The drowning prevention system of claim 16, further comprising activating the vibration motor when the moisture sensor determines that the housing is submerged in liquid.

18. The drowning prevention system of claim 13, further comprising sending a location determined by a GPS unit disposed within the housing when the alert signal is sent.

19. The drowning prevention system of claim 13, further comprising sending the alert signal to emergency services.

20. The drowning prevention system of claim 13, further comprising displaying a current heartrate detected by the heartbeat sensor on a display disposed on the front side of the housing.
